(19) United States
(12) Patent Application Publication
(10) Pub. No.: US 2025/0253063 A1
(43) Pub. Date: Aug. 7, 2025

(54) CLINICALLY TRAINED ARTIFICIAL INTELLIGENCE ADVANCED TUMOR MANAGEMENT SYSTEM

(71) Applicant: The Board of Regents of The University of Oklahoma, Norman, OK (US)

(72) Inventors: Dee Wu, Edmond, OK (US); Katherine Morris, Portland, OR (US); Anthony Alleman, Edmond, OK (US); Kristen V. Squires, Edmond, OK (US); Evan J. Fowle, Oklahoma City, OK (US); J. Spencer Thompson, Oklahoma City, OK (US); Jennifer Holter-Chakrabarty, Oklahoma City, OK (US)

(73) Assignee: The Board of Regents of the University of Oklahoma, Norman, OK (US)

(21) Appl. No.: 18/854,412

(22) PCT Filed: Apr. 5, 2023
(86) PCT No.: PCT/US2023/017547

§ 371 Date: Oct. 4, 2024

Related U.S. Application Data
(60) Provisional application No. 63/327,785, filed on Apr. 5, 2022.

(51) Int. Cl. G16H 80/00; G06N 20/00; G16H 10/60; G16H 30/20; G16H 40/67; G16H 50/70; G16H 70/00

(52) U.S. Cl. CPC G16H 80/00; G06N 20/00; G16H 10/60; G16H 30/20; G16H 40/67; G16H 50/70; G16H 70/00

(57) ABSTRACT

An artificial intelligence enabled advanced treatment management system (AI-ATMS) is configured to improve inter-actions and outcomes from a multidisciplinary patient care team. The AI-ATMS includes advanced integration with an artificial intelligence system with access to federated databases. The AI-ATMS is configured for the real-time engagement with the patient-treatment team in a manner that provides the patient-treatment team with context-related data elements, while also permitting the patient-treatment team with the ability to train the AI-ATMS during the engagement. The AI-ATMS may be for treating a cancer or other medical condition.

### RELATED APPLICATIONS

This application claims the benefit of U.S. Provisional Patent Application Ser. No. 63/327,785 filed Apr. 5, 2022 entitled, “Clinically Trained Artificial Intelligence Advanced Tumor Management System,” the disclosure of which is herein incorporated by reference as if fully set forth in this specification.

### BACKGROUND

Modern cancer treatment programs often follow a multidisciplinary approach in which subject matters experts work together as a team to provide patient care. In many cases, a group of subject matter experts, sometimes referred to as a “tumor board,” meets on a periodic basis to discuss the diagnosis and treatment of cancer patients. The board's goal is to determine the best possible cancer treatment and care plan for each cancer patient.

Collecting and organizing information from various subject matter experts can be challenging. A variety of software programs have been developed in an attempt to better coordinate the information available from medical sources and experts. The early attempts at developing systems that can improve tumor management are still evolving and currently lack attention to several subspecialties (including, but not limited to, surgery, oncology, radiation therapy, pathology, radiology, nurse navigators, and patients). Limitations in existing software systems may be the result of a lack of understanding among the software vendors about the cross-management issues that happen in medical institutions, with current vendors instead relying on single-subspecialty practitioners to provide critical patient care information.

Various attempts have also been made to engage artificial intelligence technologies (“A.I.”) to address the shortcomings of earlier tumor board technology platforms. While there are a variety of reasons these prior attempts failed to develop an advanced tumor management system that leverages strengths and benefits of A.I., a common failure is the inability to “train” the chosen A.I. to be context aware. Context awareness in the tumor management field refers to the ability of an A.I. to perceive the meaning of the information available in a patient's treatment records at a sufficient level to provide value to the patient's treatment team.

The level of context awareness needed to provide value is dependent upon the particular application of A.I. in the tumor management software. To develop the appropriate level of context awareness, an A.I. must be “trained” to recognize the relationships between a variety of data elements collected from the patient's treatment records, treatment team notes, medical research, etc. Such context awareness training can provide value to the treatment team and patient, as a system including such an A.I. can properly highlight treatment options, provide alternative diagnoses, and alert treatment team members to potential issues or challenges to the proposed (or already ordered) treatment protocols. Ideally, this value is provided at a time where such options, diagnoses, and alerts can be acted upon for the benefit of the patient.

Providing timely value to the treatment team is only achievable if the A.I. system is properly trained to recognize which relationships between data elements are meaningful, and which are not. Prior efforts at training A.I. systems in this field have failed because, in part, training an appropriate level of context awareness requires an excessive amount of time from human technicians and subject matter experts outside of the clinical environment. Because the subject matter experts in this field are highly educated physicians, their time is valuable, is often very limited, and thus as a result, finding a sufficient number of qualified subject matter experts to spend the requisite time to train the A.I. is difficult. Also, when such experts are identified, their schedules are often too congested to permit them to take time away from their respective medical practices to train the A.I. systems.

Prior attempts at developing an A.I. system in this field have also failed because the A.I. engines have been trained outside of the clinical context. In addition to removing the subject matter experts from their clinical responsibilities, training an A.I. system outside of the clinical context deprives the A.I. engine of valuable data points that only arise during treatment of patients in real-world circumstances. Without such data points, prior efforts to develop at A.I. system have failed to achieve sufficient context awareness to provide value to the treatment team, while also requiring the removal of the limited number of qualified subject matter experts from the clinic. Accordingly, there is a need to develop an AI-ATMS that can be trained to be context aware in an existing clinical setting. It is to these and other deficiencies in the prior art that the disclosed embodiments are directed.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness.

FIG. 1 is an overview diagram depicting a treatment team training an AI-ATMS in a clinical setting.

FIG. 2 is a wireframe diagram of a graphical user interface to the application interface of the AI-ATMS of FIG. 1.

FIG. 3 provides a process flow chart for using and training the AI-ATMS of FIG. 1.

FIG. 4 is a wireframe diagram of a first example of a discipline specific portal for the AI-ATMS of FIG. 1.

FIG. 5 is a wireframe diagram of a second example of a discipline specific portal for the AI-ATMS of FIG. 1.

FIG. 6 is a wireframe diagram of a third example of a discipline specific portal for the AI-ATMS of FIG. 1.

FIG. 7 is a wireframe diagram of a fourth example of a discipline specific portal for the AI-ATMS of FIG. 1.

### DETAILED DESCRIPTION

The present disclosure is generally directed to the development and use of an artificial intelligence enabled advanced treatment management system 100 (an AI-ATMS). In at least one embodiment, the AI-ATMS is directed to a tumor (i.e., cancer) management system. In exemplary embodiments, the AI-ATMS 100 is well-suited to provide a comprehensive platform for a multidisciplinary patient-treatment team (sometimes referred to as a “tumor board”), with advanced integration to an artificial intelligence system, and access to federated databases. In exemplary embodiments, the AI-ATMS 100 is configured for real-time engagement with the patient-treatment team in a manner that provides the patient-treatment team with context-related data elements, while also permitting the patient-treatment team with the ability to train the AI-ATMS 100 during the engagement.

Before describing various embodiments of the present disclosure in more detail by way of exemplary description, examples, and results, it is to be understood that the embodiments of the present disclosure are not limited in application to the details of methods and apparatus as set forth in the following description. The embodiments of the present disclosure are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that certain embodiments of the present disclosure can be practiced without these specific details. In other instances, features which are well known to persons of ordinary skill in the art have not been described in detail to avoid unnecessary complication of the description.

Unless otherwise defined herein, scientific and technical terms used in connection with the embodiments of the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which embodiments of the present disclosure pertain. All patents, published patent applications, and non-patent publications referenced in any portion of this application are herein expressly incorporated by reference in their entirety to the same extent as if each individual patent or publication was specifically and individually indicated to be incorporated by reference.

While the methods and apparatus of the embodiments of the present disclosure have been described in terms of particular embodiments, it will be apparent to those of skill in the art that variations may be applied thereto and, in the steps, or in the sequence of steps of the methods described herein without departing from the spirit and scope of the inventive concepts. All such similar substitutes and modifications apparent to those skilled in the art are deemed to be within the spirit and scope of the systems as defined herein.

As utilized in accordance with the methods and apparatus of the embodiments of the present disclosure, the following terms, unless otherwise indicated, shall be understood to have the following meanings.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error. Further, in this detailed description, each numerical value (e.g., time or frequency) should be read once as modified by the term “about” (unless already expressly so modified), and then read again as not so modified unless otherwise indicated in context. The use of the term “about” or “approximately” may mean a range including ±0.5%, or ±1%, ±2%, or ±3%, or ±4%, or ±5%, ±6%, or ±7%, or ±8%, or ±9%, or ±10%, or ±11%, or ±12%, or ±13%, or ±14%, or ±15%, or ±25% of the subsequent number unless otherwise stated.

As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, or at least 90% of the time, or at least 95% of the time, or at least 98% of the time.

Features of any of the embodiments described herein may be combined with any of the other embodiments to create a new embodiment. As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50. Similarly, fractional amounts between any two consecutive integers are intended to be included herein, such as, but not limited to, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, and 0.95. For example, the range 3 to 4 includes, but is not limited to, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, and 3.95. Thus, even if specific data points within the range, or even no data points within the range, are explicitly identified or specifically referred to, it is to be understood that any data points within the range are to be considered to have been specified, and that the inventors possessed knowledge of the entire range and the points within the range.

Reference to a series of ranges includes ranges which combine the values of the boundaries of different ranges within the series. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range.

Thus, to further illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure.

The term “clinical” refers to the medical observation, examination, and treatment of actual patients.

The term “medical condition” as used herein refers to a condition which requires medical treatment to be resolved, including but not limited to, diseases and conditions such as cancer, cardiovascular disease, stroke, limb damage, brain damage or disease, organ damage, organ disease, a chronic inflammatory disease, a chronic immune disease, an infection, a genetic disease, a mental disorder, a diabetic disease, and chronic pain.

“Treatment” refers to therapeutic treatments. A successful treatment outcome can lead to a “therapeutic effect,” or “benefit” of ameliorating, decreasing, reducing, inhibiting, suppressing, limiting, controlling, or preventing the occurrence, frequency, severity, progression, or duration of the condition, or consequences of the condition in a patient.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

The present disclosure will now be discussed in terms of several specific, non-limiting, examples, and embodiments. The examples described below, which include particular embodiments, will serve to illustrate the practice of the present disclosure, it being understood that the particulars shown are by way of example and for purposes of illustrative discussion of particular embodiments and are presented in the cause of providing what is believed to be a useful and readily understood description of procedures as well as of the principles and conceptual aspects of the present disclosure.

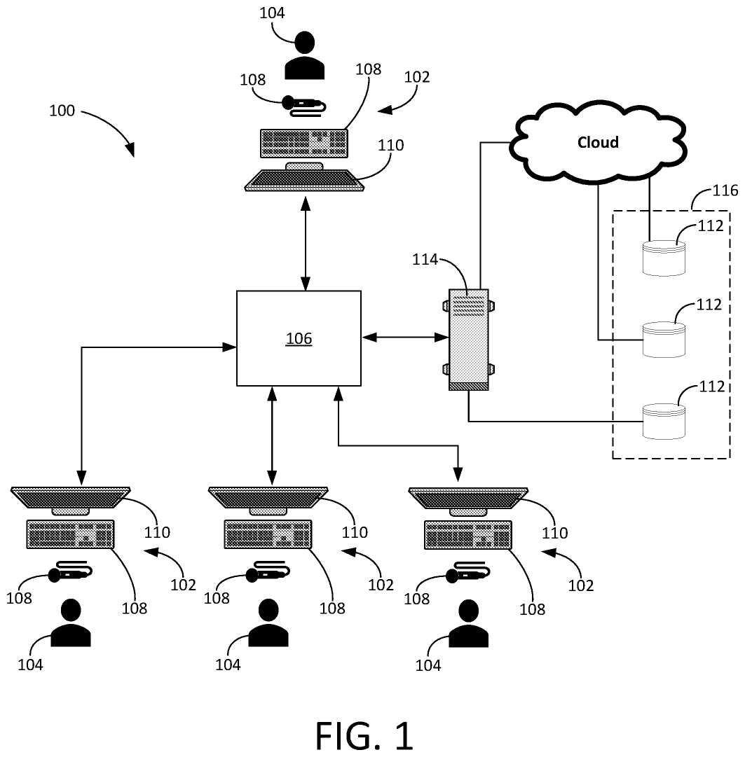

Turning to FIG. 1, shown therein is an embodiment of the AI-ATMS 100 in which the AI-ATMS 100 includes a plurality of user interfaces 102, which are designed for use by participants 104 comprising a multidisciplinary patient care team. The participants 104 can be physicians, surgeons, nurses, technicians, technologists, physical therapists, researchers, subject matter experts, office personnel, administrators, and other members of a multidisciplinary patient care team. The participants 104 may include the patient, who can be provided with restricted access to the AI-ATMS 100. In some embodiments, the participants 104 are provided with credentials that are used by the AI-ATMS 100 to provide or restrict access to certain information and to tailor the output of the AI-ATMS 100 to meet the qualifications or training of each participant 104. Although the systems and methods disclosed herein are designed for use in connection with the AI-ATMS 100, it will be appreciated that the same systems and methods could also be used in other situations in which a members of a team need to work together to achieve common goals using a collaborative platform.

The interfaces 102 can be personal computers or mobile computing devices such as smartphones that are configured to run or access a common application 106. In exemplary embodiments, the interfaces 102 include input devices 108 and output devices 110. The input devices 108 can include, for example, microphones, cameras, and keyboards. The output devices 110 can include, for example, computer monitors, display screens, printers, speakers, or other output devices. The application 106 is a computer program that is designed to exchange and display information between the interfaces 102, one or more databases 108, and an artificial intelligence component 110. The interfaces 102 can be located in a common facility (e.g., a hospital) or in different locations. For example, one or more of the participants 104 can be located at home or at a remote office, while the other participants 104 are together in a common meeting room. The application 106 can be configured to run on each interface 102, or on one or more remote computers that are accessed by the interfaces 102 in a client-server relationship.

It will be understood by those in the tumor management field that clinical meetings between participants 104 (i.e., a tumor board) take place periodically during a patient's treatment. These clinical meetings are often scheduled at the hospital (or another cancer treatment facility) and take place in a single room. For in-person meetings, the input devices 108 include one or more microphones positioned and configured to capture the audio from the participants' discussion during the meeting. It will be further understood, however, that modern teleconferencing and videoconferencing technologies such a Zoom, Go To Meeting, and Microsoft Teams are widely available and have been deployed to hospitals and other treatment centers to a sufficient degree that appropriate clinical meetings can take place without the participants 104 being present in the same room. In such circumstances, where attendance of at least once of the participants 104 is remote, the input devices 108 can include headsets, microphones and cameras configured to capture the audio and video from the remote participant 104.

The interfaces 102 are connected to one or more databases 112. The databases 112 can include general medical data, biographical and medical information about the patient, research about the patient's condition, treatment options, and prognoses. In some embodiments, the one or more databases 112 are functionally aggregated as federated databases 116, which can be accessed by the participants 104 or the application 106 through unified calls or requests for information. The extent to which the participants 104 can access the federated databases 116 can be controlled by access credentials assigned to each participant 104, as carried by proxy through the application 106. The databases 112 can, for example, include standard and emerging treatment protocols, standards of care, clinical data, patient outcome data, genetic-based treatment and outcome data, and pharmaceutical and medicinal information.

The databases 112 can be a collection of private and public health-related databases from around the world. The databases 112 can include data and factors regarding any number of diseases, health conditions and treatments. The databases 112 can, for example, include data on comorbidities, barriers to care, hypertension, autoimmune diseases and responses, kidney disease, patient transportation-related factors, patient frailty, obesity (BMI), income disparity, racial disparities, smoking, emergency room access, EHR/PACS system access, internet access, lung screening rates, births (parity=number of children), number of pregnancies carried to term, social support systems, breast screening rates, colon screening, insurance, mental health, educational levels, health literacy, environmental chemicals, accidents, language barriers, medications, access to generics (and grant money for generics), diabetes, rural-related access and health considerations, alcohol and drug dependency rates, access and history of rehabilitation services, cardiac health, car accidents, access to narcotic rescue medications, access to food pantries, access to radiation and chemotherapy therapy centers, access to routine medical care, genetic factors, surgical history, blood pressure rates, pediatric developmental information, and family history of medical conditions.

The interfaces 102 are also connected to an artificial intelligence component 114 through the application 106 (as depicted in FIG. 1). Alternatively, each interface may be connected to the artificial intelligence component 114 directly. In exemplary embodiments, the artificial intelligence component 114 is a computer-implemented tool that is programmed to observe the meeting between the participants 104, record audio, video or other inputs from the participants 104, parse the input from the participants 104 into literal text, recognize clinical data elements within the text, query the databases 112 (or federated database 116) with the clinical data elements, generate context-related data elements based on the intersection of the clinical data elements and medical data from the databases 112, and provide the context-based data elements to the participants 104 through the application 106 and output devices 110.

It will be understood by those skilled in the art that protocols exist that permit the one or more recordings produced by the interfaces 102 to be transmitted to the artificial intelligence component 114 in real time or near real time. In some embodiments, the artificial intelligence component 114 is configured to transcribe the recorded audio from the interfaces 102 into multiple languages. For example, the artificial intelligence component 114 can be configured to automatically transcribe the recorded English-language audio from the participants into English, French, German, Spanish, Japanese, Korean, Chinese, Hindi, and/or Bengali text or other languages so the clinical data elements can be searched in databases 212 that include reference data elements in multiple languages. The artificial intelligence component 114 can also be configured to translate search results retrieved from databases 212 from a second language back into the first language used by the participants 104. The language translation function of the artificial intelligence component 114 also permits participants 104 to engage in the meeting from multiple countries using multiple languages (e.g., a participant 104 in the United States speaking English engaging with a participant in German speaking German). It will be appreciated that, in some embodiments, the artificial intelligence component 114 and databases 112 will communicate with one another and peripheral systems more directly through machine-level language or computer-specific communication protocols, without translating the communication into human-readable language.

In broad terms, the artificial intelligence component 114 can be configured or structured as a trainable artificial engine with suitable programming to accurately identify clinical data elements based on input from the participants 104, retrieve context-based data from the databases 112, and then provide the context-based data elements to the participants 104 in real-time. As used herein, the term “clinical data element” refers to information or data produced by the participants 104. The term “reference data element” refers to medical and other data contained within the databases. The term “context-based data element” refers to data produced by the artificial intelligence component 114 based on a relationship identified by the artificial intelligence component 114 between a clinical data element and reference data. As used herein, the term “real-time” refers to a response from a system or process step carried our promptly, immediately, without intervening intentional delays, or within a time frame that does not disrupt the exchange of information between participants 104 in the clinical meeting. For example, in the context of the exemplary embodiments, the term “real-time” can refer to the time required by the artificial intelligence component 114 to listen to discussions by participants 104 in a clinical meeting, transcribe the audio from those discussions, use the transcription as clinical data elements to look through the databases 212 for relevant information, and return corresponding context-related data elements to the participants 104 during the same meeting.

The artificial intelligence component 114 can retrieve information from the databases 112 through direct connections or through a cloud API such as REST, SOAP, or any other available communication method that permits the artificial intelligence component 114 to (i) send queries based on the input provided by the participants 104 whether typed, spoken, or otherwise entered into the application 106; and (ii) receive responsive, context-appropriate data from the databases 112. It will be appreciated that the artificial intelligence component 114 can include multiple interconnected modules. For example, the transcription function can be performed by a discrete software application within the artificial intelligence component 114, while the context-based search and retrieval systems are managed by one or more other distinct modules within the artificial intelligence component 114. The artificial intelligence component 114 can be configured to adapt its processes to new environments through meta-learning and learn-to-learn systems and programming.

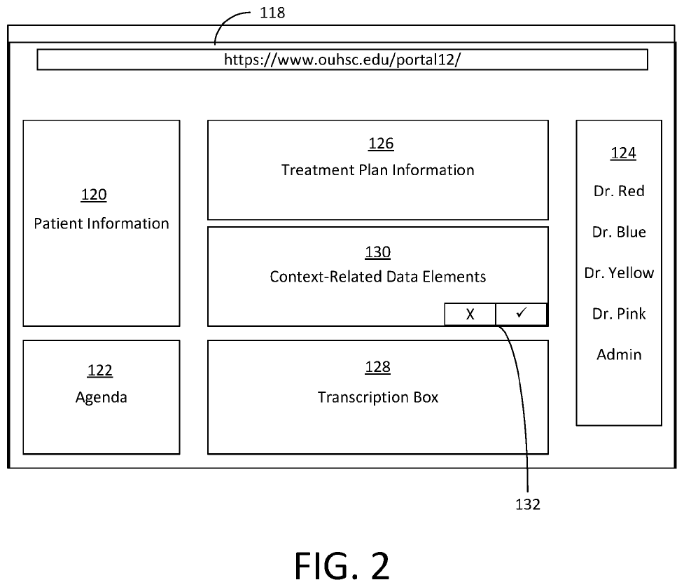

For example, FIG. 2 depicts a simplified engagement page 118 from the application 106, which could be used by the participants 104 during a meeting in which the AI-ATMS 100 is used. The page 118 can be displayed, for example, through a standard secure browser running on the interface 102 and connected to the application 106. In this embodiment, the page 118 includes a patient information box 120, a meeting agenda box 122, a roster box 124 of the participants 104, and a treatment plan overview box 126. In each case, the boxes are simply sub-windows or fields within the engagement page 118 that retrieve and display appropriate information.

The engagement page 118 also includes a transcription box 128 that provides the transcription from the artificial intelligence component 114 of the audio recordings captured from the participants 104. The transcription box 128 can be configured to allow the participants 104 to search or scroll through comments made earlier during the meeting, or during previous meetings. The engagement page 118 also includes an AI-generated output box 130. The AI-generated output box 130 can be a standalone box (as depicted in FIG. 2) or integrated into other fields or boxes within the engagement page 118. The AI-generated output box 130 that displays context-related data elements produced by the artificial intelligence component 114 in response to queries run by the artificial intelligence component 114 based on the transcribed input from the participants 104.

In exemplary embodiments, the context-related data elements are displayed on the engagement page 118 in real time or near real-time. If, for example, one of the participants 104 suggests a new medication protocol for the patient that might cause an undesirable interaction with another medication already prescribed to the patient, that suggestion is transcribed by the artificial intelligence component 114 and used as the basis for a contextual database search. The artificial intelligence component 114 can identify the potential adverse interaction and post a warning or other context-related data element to the engagement page 118 for review and consideration by the participants 104.

The artificial intelligence component 114 can be trained to produce context-related data elements on a wide range of topics. In some embodiments, the artificial intelligence component 114 is configured to produce context-related data elements that include predicted risks based on age, fitness, and other patient lifestyle attributes. In other embodiments, the artificial intelligence component is configured to determine context-related data elements that include potential alternative therapies that should be considered by the participants 104.

The AI-generated output box 130 can also include a participant feedback module 132 that allows the participants 104 to confirm, reject or otherwise respond to the context-related data elements posted by the artificial intelligence component 114 to the engagement page 118. Based on this feedback, the artificial intelligence component 114 can update references in the databases 112 or the alternatively discrete modules of the artificial intelligence component 114 to indicate whether the generated context-related data element was appropriate based on the expert opinions of the participants 104. This allows the participants 104 to actively train the artificial intelligence component 114 in real time during the meeting. By training the artificial intelligence component 114 in this manner, the participants 104 are not required to leave the clinical environment in which they serve their patients.

In one sense, this can be interpreted as allowing each participant 104 to vote on whether each context-related data element created by the artificial intelligence component 114. Upon receiving sufficient feedback regarding a particular context-related data element to determine that a consensus has been reached, the artificial intelligence component 114 can generate a confirmed context data element, and display that on any of the interfaces in the plurality of interfaces 102. Based on the feedback provided by the participants 104, the AI-ATMS 100 can discard faulty or inaccurate correlations drawn by the artificial intelligence component 114, while promoting or confirming those correlations that the participants deem accurate and appropriate.

In some embodiments, the context-related data elements produced by the artificial intelligence component 114 are topical, relevant or appropriate to only some of the participants 104 on the multidisciplinary team. Given the background and credentials of each participant 104, the artificial intelligence component 114 can select which participants 104 should be shown a specific context-related data element. The artificial intelligence component 114 can be configured to seek approval from qualified participants 104 before sharing the context-related data elements with other (non-qualified) participants 104.

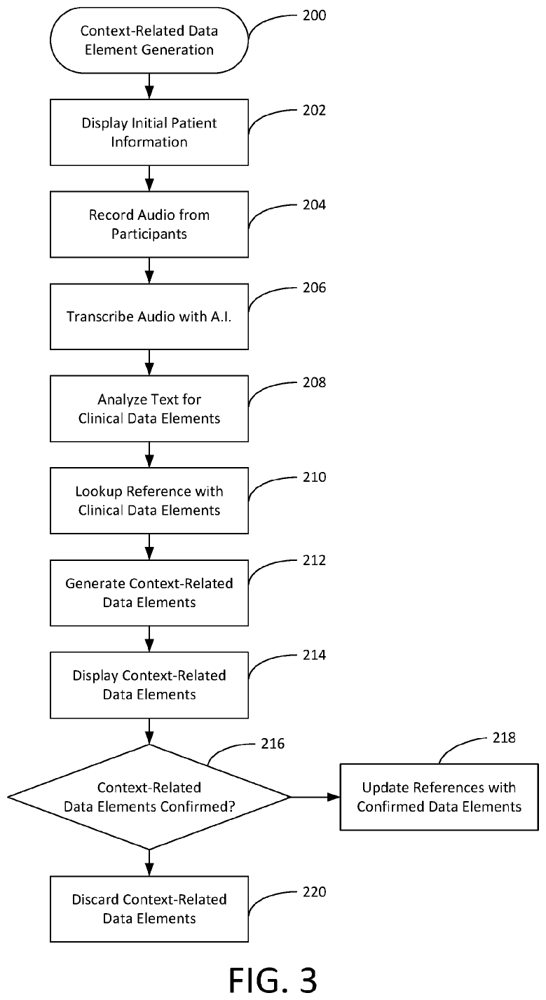

Turning to FIG. 3, shown therein is a process flow chart illustrating one method 200 for using the AI-ATMS 100 in a clinical meeting. When the participants 104 meet in a clinical meeting to discuss the treatment of a patient, the AI-ATMS 100 is engaged by activating the application 106 on the interfaces 102. The artificial intelligence component 114 can establish connections to the interfaces 102 and the databases 112 when the AI-ATMS 100 is activated.

At step 202, the application 106 displays the initial information on the engagement page 118, which can include the background biographical information about the patient, and diagnoses and current treatments. Each participant 104 interacts with their respective interface 102 and reviews the patient information displayed by the application 106 on the output devices 110. As the participants 104 discuss the case, the input devices 108 gather input from the participants. At step 204, the audio from the participants 104 is recorded by the input devices 108 and the audio is transcribed into literal text at step 206. At step 208, the artificial intelligence component 114 parses the literal text from the transcription into conceptual text. The artificial intelligence component 114 includes or accesses language processing programs to recognize clinical data elements from the transcribed text.

At step 210, the artificial intelligence component 114 uses the clinical data elements produced at step 208 and queries the databases 112 for reference data elements and information that corresponds to the various concepts recognized by the artificial intelligence component 114. The responses to the queries are then used by the artificial intelligence component 114 to identify potential relationships between the patient information, clinical data elements, and reference data elements. Upon identifying such a relationship or intersection, the artificial intelligence component 114 produces a context-related data element at step 212. The artificial intelligence component 114 displays the context-related data element on the interfaces 102 at step 214 for review by the participants 104.

At step 216, each participant 104 can choose to provide feedback through their respective interface 102 to the artificial intelligence component 114. The participants can, for example, use the feedback module 132 to indicate whether the context-related data elements are appropriate to the discussion, accurate, and suitable for use in future use by the AI-ATMS 100. The manner in which the participants 104 provide feedback may differ based upon the particular embodiment of the application 106.

If the context-related data elements are confirmed at step 216, the artificial intelligence component 114 can update the databases 212 with a confirmed relationship between the clinical data element produced by the participants 104 and the context-related data elements generated by the artificial intelligence component 114. If, on the other hand, the participants 104 reject the context-related data elements as inaccurate or inappropriate to the underlying clinical data, the context-related data elements are discarded at step 220. The artificial intelligence component 114 can update the databases 112 to indicate the inaccurate or inappropriate context-related data elements. Confirming or rejection the context-related data elements allows the participants 104 to efficiently train the artificial intelligence component 114.

The artificial intelligence component 114 can be configured to look for digital twinning examples based on the clinical data elements produced by the participants 104. Based on the clinical data elements, the artificial intelligence component 114 can look through appropriate databases 112 for cases involving similar patients and diagnoses (often referred to as a “digital twin case”). The artificial intelligence component 114 can then generate context-related data elements based on the specific data elements associated with the digital twin case encountered in the databases 112, including treatment options, contraindications and prognoses for treatment protocols. The artificial intelligence component 114 can also be configured to articulate the degree to which the patient matches one or more of the digital twins encountered in the databases 112.

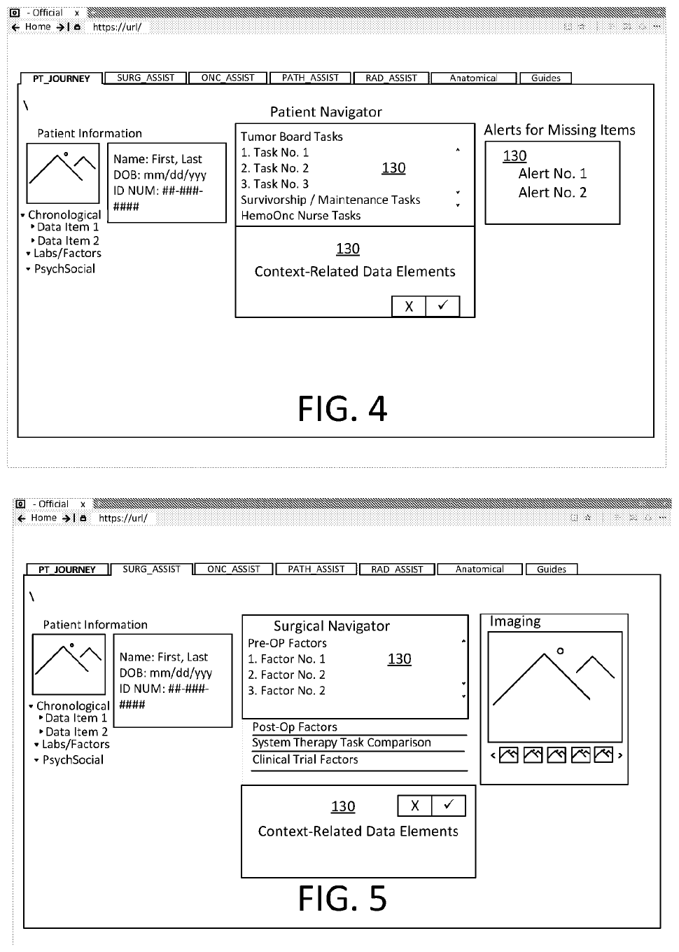

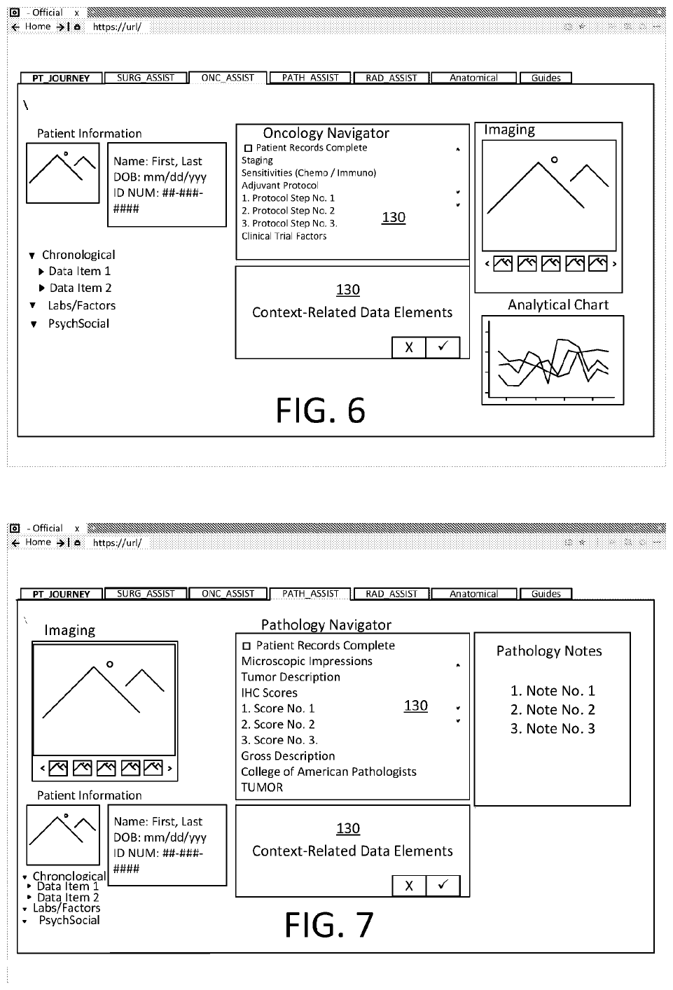

In some embodiments, the application 106 includes a series of specialty modules 134 that are designed to provide information to specific participants 104 based on their training, credentials and specialty. FIGS. 4-7 provide examples of specialty modules 134 that are designed specifically for the patient (FIG. 4), the surgical team (FIG. 5), the oncology team (FIG. 6), and the pathology team (FIG. 7). Other specialty modules 134 are used to exchange information with participants 104 with other specialties. Each specialty module 134 is designed to provide subject-matter specific information to the participants 104, which may include context-related data elements produced by the artificial intelligence component 114.

In FIG. 4, for example, the artificial intelligence component 114 can provide context-related data elements for “Tumor Board Tasks” and “Alerts” for missing information from the patient. In FIG. 5, the artificial intelligence component 114 can identify context-related data elements that include pre-operative risk factors to be considered by the surgical team. In FIG. 6, the artificial intelligence component 114 can provide the oncology team with context-related data elements that relate to chemotherapy recommendations and contraindications. The artificial intelligence component 114 can also be configured to identify particular pathological and genetic information as context-related data elements to the pathology team, as illustrated in FIG. 7. Each page within the application 106 can be configured to display patient information, page-specific navigation menus, pending or completed tasks, and other general or subject-matter specific information.

Importantly, the AI-ATMS 100 provides a system in which the artificial intelligence component 114 provides context-related data elements to the participants 104 in real time based on the clinical discussion occurring at that time. This allows the participants 104 to promptly identify relevant information from a vast array of resources in the databases 112 in a manner that could not be replicated or approximated the artificial intelligence component 114. Providing near-instantaneous context-related data elements based on the intersection of automatically determined clinical data elements and reference data elements could not be accomplished by hand. Conventional research involves hours of manual research using search queries that may be compromised by terminological differences, imprecise language and foreign language differences. Additionally, because the AI-ATMS 100 includes an efficient process for training the artificial intelligence component 114 based on feedback from the participants 104, the accuracy and scope of the AI-ATMS 100 will expand through use.

In some embodiments, the present disclosure is thus directed to a computer-enabled process for conducting a clinical meeting with a plurality of participants discussing a patient undergoing treatment for a medical condition. The process begins with the step of providing an interface for each of the plurality of participants, wherein each of the plurality of interfaces is configured to record a clinical discussions of the corresponding participant. Next, the process includes the steps of recording the clinical discussion of the plurality of participants with the plurality of interfaces, automatically transcribing the recorded clinical discussions into text in real time, displaying the transcribed text on the interface in real time and automatically identifying clinical data elements from the text with an artificial intelligence component in real time. While the participants are discussing the clinical data elements, the artificial intelligence component automatically accesses a database that includes reference data elements, identifies relational intersections between the clinical data elements and the reference data elements, and produces a context-related data element from the relational intersection between the clinical data elements and the reference data elements. The process continues by automatically displaying the context-related data element on the interface in real time during the corresponding clinical discussion. To train the artificial intelligence component 114, the process includes the step of soliciting from the plurality of participants a decision regarding the context-related data element. The decision may be, for example an approval, disapproval, modification, or deferment regarding the context-related data element.

In other embodiments, the present disclosure is directed to an advanced tumor management system for use by a multidisciplinary team of participants in a clinical meeting. The system includes a software application and an interface. The interface includes an input device configured to record discussion of the participants and an output device. The interface is configured to access the software application. The system also includes a database that includes reference data elements and an artificial intelligence component connected to the database and to the interface. The artificial intelligence component is configured to determine clinical data elements from the recorded discussion of the participants, generate context-related data elements based on determined relationships between the clinical data elements and the reference data elements, and display the context-related data elements on the output device.

Thus, the embodiments of the present disclosure are well-adapted to carry out the objects and attain the ends and advantages mentioned above, as well as those inherent therein. While the AI-ATMS 100 has been described and illustrated herein by reference to particular non-limiting embodiments in relation to the drawings attached thereto, various changes and further modifications, apart from those shown or suggested herein, may be made therein by those of ordinary skill in the art, without departing from the spirit of the inventive concepts.

1. A computer-enabled process for conducting a clinical meeting with a plurality of participants discussing a patient undergoing treatment for a medical condition, the process comprising the steps of:
providing an interface for each of the plurality of participants, wherein each of the plurality of interfaces includes an input device that is configured to record a clinical discussion of the corresponding participant;
recording the clinical discussion of the plurality of participants with the input devices on the plurality of interfaces;
automatically transcribing the recorded clinical discussion into text in real time;
displaying the transcribed text on the interface in real time;
automatically identifying clinical data elements from the text with an artificial intelligence component in real time;
while the participants are discussing the clinical data elements, automatically instructing the artificial intelligence component to access a database that includes reference data elements;
automatically identifying with the artificial intelligence component relational intersections between the clinical data elements and the reference data elements;
automatically producing a context-related data element from the relational intersection between the clinical data elements and the reference data elements;
automatically displaying the context-related data element on a display screen within the interface in real time during the corresponding clinical discussion; and
soliciting from the plurality of participants a decision regarding the context-related data element, wherein at least one of the participants responds with the decision regarding the context-related data element.

2. The process of claim 1, further comprising the step of automatically updating the databases with the decision regarding the context-related data element.

3. The process of claim 2, wherein the decision regarding the context-related data element is an approval, disapproval, deferment, or modification of the context-related data element.

4. The process of claim 1, wherein the step of automatically identifying relational intersections between the clinical data elements and the reference data elements further comprises automatically identifying a digital twin for the patient within the database.

5. The process of claim 4, further comprising the step of displaying information about the digital twin on the plurality of interfaces.

6. The process of claim 1, wherein the step of automatically transcribing the recorded clinical discussion further comprises automatically transcribing the recorded clinical discussion into at least one language other than English.

7. The process of claim 1, wherein the medical condition is selected from the group consisting of cancer, cardiovascular disease, stroke, limb damage, brain damage or disease, organ damage, organ disease, a chronic inflammatory disease, a chronic immune disease, an infection, a genetic disease, a mental disorder, a diabetic disease, and chronic pain.

8. A process for developing an artificial intelligence enabled advanced treatment management system that includes an artificial intelligence component, a plurality of databases accessible by the artificial intelligence component, and a plurality of computer interfaces for use by participants in a clinical meeting, the process comprising the steps of:
providing each participant with a corresponding one of the plurality of computer interfaces;
recording the clinical discussion of the participants with one or more microphones connected to the plurality of computer interfaces;
automatically transcribing the recorded clinical discussion into text in real time;
displaying the transcribed text on a display screen within the plurality of computer interfaces in real time;
automatically identifying clinical data elements from the text with the artificial intelligence component in real time;
while the participants are discussing the clinical data elements, automatically instructing the artificial intelligence component to access a database that includes reference data elements;
automatically identifying with the artificial intelligence component relational intersections between the clinical data elements and the reference data elements;
automatically producing a context-related data element from the relational intersection between the clinical data elements and the reference data elements;
automatically displaying the context-related data element on the display screens of the plurality of interfaces in real time during the corresponding clinical discussion;
soliciting from the participants a decision regarding the context-related data element, wherein at least one of the participants responds with the decision regarding the context-related data element; and
updating the artificial intelligence component during the clinical meeting by indicating the decision regarding the context-related data element.

9. The process of claim 8, wherein the advanced treatment management system is for the treatment of a medical condition selected from the group consisting of cancer, cardiovascular disease, stroke, limb damage, brain damage or disease, organ damage, organ disease, a chronic inflammatory disease, a chronic immune disease, an infection, a genetic disease, a mental disorder, a diabetic disease, and chronic pain.

10. The process of claim 8, wherein the decision regarding the context-related data element is an approval, disapproval, deferment, or modification of the context-related data element.

11. An advanced treatment management system for use by a multidisciplinary team of participants in a clinical meeting, the system comprising:
a software application;
an interface, wherein the interface comprises:
an input device configured to record discussion of the participants;
an output device; and
wherein the interface is configured to access the software application;
a database, wherein the database includes reference data elements; and
an artificial intelligence component connected to the database and to the interface, wherein the artificial intelligence component is configured to determine clinical data elements from the recorded discussion of the participants, generate context-related data elements based on determined relationships between the clinical data elements and the reference data elements, and display the context-related data elements on the output device.

12. The advanced treatment management system of claim 11, wherein the advanced treatment management system is for the treatment of a medical condition selected from the group consisting of cancer, cardiovascular disease, stroke, limb damage, brain damage or disease, organ damage, organ disease, a chronic inflammatory disease, a chronic immune disease, an infection, a genetic disease, a mental disorder, a diabetic disease, and chronic pain.
